(19) United States
(12) Patent Application Publication
(10) Pub. No.: US 2025/0366915 A1
(43) Pub. Date: Dec. 4, 2025

(54) Infusion Module For An Electrosurgical Instrument

(71) Applicant: Stryker Corporation, Portage, MI (US)

(72) Inventors: Beau Michael Kidman, Kalamazoo, MI (US); Elizabeth J. Fisher, Phoenix, AZ (US); Phillip Bryce Michaelson, Kalamazoo, MI (US); Keven L. Chambers, Byron Center, MI (US)

(73) Assignee: Stryker Corporation, Portage, MI (US)

(21) Appl. No.: 18/875,359

(22) PCT Filed: Jun. 16, 2023
(86) PCT No.: PCT/US2023/025541

§ 371 Date: Dec. 16, 2024

Related U.S. Application Data
(60) Provisional application No. 63/352,686, filed on Jun. 16, 2022.

(51) Int. Cl. A61B 18/16; A61B 18/00

(52) U.S. Cl. CPC A61B 18/16; A61B 2018/00023; A61B 2018/00565; A61B 2018/00577

(57) ABSTRACT

A medical device for delivering a liquid to an electrosurgical instrument. A plunger is movably disposed and biased within a fluid reservoir. The plunger includes a first colored portion and a second colored portion. The first colored portion is viewable through the window with the fluid reservoir containing a first volume, and movement of the plunger is configured to expose the second colored portion as the fluid reservoir discharges liquid. A housing of the device may include a first shell including a female snap and a second shell including a male snap. The female snap includes a female annular projection, a female snap undercut, and a central locking feature. The central locking feature is received within a male annular projection to extend from the first shell to an axial position closer to a distal end of the female annular projection than the female snap undercut.

### PRIORITY CLAIM

This application claims priority to and all the benefits of U.S. Provisional Patent Application No. 63/352,686 filed Jun. 16, 2022, the entire contents of which are hereby incorporated by reference.

### BACKGROUND

Radiofrequency (RF) energy is commonly utilized to ablate diseased tissue to treat pain or pathology. The tissue may be sensory nerves, intraosseous nerves, or intraosseous tumors, among other anatomic structures. Conventionally, an electrode is coupled to an electrosurgical console, and the RF energy is conducted from the electrode to the tissue across an electrode-tissue interface to create a lesion at the treatment site. For intraosseous tumors, the RF energy often heats the tissue to at least 90° C. (194° F.) to destroy the cells of the tumor. Due to the high temperatures of the treatment site, infusion of a fluid, such as saline, to the treatment location may limit charring by improving conductivity across the electrode-tissue interface. Thus, providing a continuous and consistent infusion of fluid to the treatment site may increase the efficacy of ablation procedures while also reducing the risks associated with such procedures.

It is known to provide cooling or irrigation fluid with an electronically-controlled fluid pump system, which may be complex in construction and cumbersome to operate. One solution is an infusion module disclosed in commonly-owned International Publication No. 2020/198150, published Oct. 1, 2020, in which a spring is biased within a housing to provide consistent delivery of the fluid to the treatment site. There remain additional opportunities for improvement to the infusion module to increase its robustness and usability.

### SUMMARY

According to a first aspect of the present disclosure, a medical device for delivering a liquid to an electrosurgical instrument includes: a housing defining a window. A fluid reservoir is disposed within the housing and configured to receive the liquid. At least a portion of the fluid reservoir is transparent. A plunger is movably disposed within the fluid reservoir and including a first colored portion and a second colored portion being of a different color than the first colored portion. A spring is disposed within the housing and configured to bias the plunger to discharge the infusion fluid to the electrosurgical instrument. The medical device is configured to be viewed by a user such that the first colored portion of the plunger is viewable through the window with the fluid reservoir containing a first volume of the liquid, while the plunger is configured to expose the second colored portion of the plunger within the window as the fluid reservoir discharges at a least a portion of the liquid to contain a second volume of the liquid that is less than the first volume.

According to a second aspect of the present disclosure, a medical device for delivering a liquid to an electrosurgical instrument includes a housing defining a window. A fluid reservoir is disposed within the housing and configured to receive the liquid. At least a portion of the fluid reservoir is transparent. A plunger is movably disposed within the fluid reservoir and including a first colored portion and a second colored portion being of a different color than the first colored portion. A spring is disposed within the housing and configured to bias the plunger to discharge the infusion fluid to the electrosurgical instrument. The medical device is configured to be viewed by a user such that the second colored portion of the plunger is viewable through the window to be indicative of low volume of the infusion fluid in which less than approximately 20% of a filled volume of the liquid remains within the fluid reservoir.

According to a third aspect of the present disclosure, a method of monitoring delivery of a liquid during an ablation procedure with a medical device is provided. A sealing head of the plunger is viewed through the window with the fluid reservoir containing a first volume of the liquid. The medical device is operated to discharge a portion of the liquid from the fluid reservoir. A first colored portion of the plunger is viewed through the window with the fluid reservoir containing a second volume of the liquid that is less than the first volume. The medical device may be further operated to discharge another portion of the liquid from the fluid reservoir. A second colored portion of the plunger is viewed through the window with the fluid reservoir containing a third volume of the liquid that is less than the first volume and the second volume.

According to a fourth aspect of the present disclosure a medical device housing is provided in which a first shell includes a female snap, and a second shell includes a male snap. The female snap includes a female annular projection extending from the first shell to a distal end, a female snap undercut extending inwardly from the female annular projection, and a central locking feature disposed within the female annular projection. The male snap includes a male annular projection extending from the second shell to a distal end and received within the female annular projection. The central locking feature is received within the male annular projection. A male snap undercut extends outwardly from the male annular projection and engages the female snap undercut. Additionally, the central locking feature extends from the first shell to an axial position closer to the distal end of the female annular projection than the female snap undercut.

According to a fifth aspect of the present disclosure, a medical device housing is provided in which a first shell including a female snap, and a second shell including a male snap. The female snap includes a female projection extending from the first shell to a first distance and defining a first void. A female snap undercut extends into the first void and located at a second distance from the first shell. A central locking feature is disposed within the first void. The male snap includes a male projection extending from the second shell and defining a second void. The male projection is received within the first void and the central locking feature is received within the second void. A male snap undercut extends away from the second void. A male snap undercut extends outwardly from the male projection and engages the female snap undercut. Additionally, the central locking feature extends from the first shell to a third distance that is greater than the second distance.

According to a sixth aspect of the present disclosure, a medical device housing is provided in which a first shell including a female snap, and a second shell including a male snap. The female snap includes a female projection extending from the first shell and defining a first void and at least one slot. A female snap undercut extends inwardly from the female projection. A central locking feature is disposed within the first void of the female projection. The male snap includes a male projection extending from the second shell and defining a second void and at least one slot. The male projection is received within the first void and the central locking feature is received within the second void. A male snap undercut extends outwardly from the male projection and engaging the female snap undercut. Additionally, the slots of are configured to permit deflection of at least one of the female projections and the male projection for the female snap to be mated with the male snap. The central locking feature extends from the first shell to an axial position configured to limit or prevent the inward deflection of the male projection with the first snap mated with the second snap.

Any of the above aspects can be combined in part or in whole with any other aspect. Any of the above aspects, whether combined in part or in whole, can be further combined with any of the following implementations, in full or in part.

In certain implementations, the second colored portion is not visible through the window with the fluid reservoir containing the first volume of the liquid. The liquid may be viewable through the window with the fluid reservoir containing the first volume of the liquid. The first colored portion and the second color portion may span a length of the window, and this is indicative that the fluid reservoir is substantially empty.

In certain implementations, the plunger further includes a sealing head being of another different color than the first colored portion and the second colored portion, and the scaling head is viewable through the window with the fluid reservoir containing a third volume of the liquid that is greater than the first volume. The third volume may be indicative that the fluid reservoir is substantially filled with the liquid. The plunger may include a support member coaxially disposed within the spring and engaging an internal shelf of the housing. The plunger may be formed from a material of a same color as the first colored portion, and wherein the second colored portion is coupled to the plunger. The window may oblong, and a length of the window may correspond to approximately six milliliters of the liquid being viewable within the fluid reservoir. The fluid reservoir may be devoid of volume markings.

In certain implementations, the device includes a port assembly defining a first inlet, a second inlet, and an outlet, and the valve. The valve may be configured to direct the liquid injected from an external source through the first inlet into the fluid reservoir, and further direct the liquid discharged from the fluid reservoir through the second inlet and through the outlet. The port assembly may engage a second internal barrier of the housing so as to resist movement of the fluid reservoir from forces from the plunger being biased by the spring. The outlet may be oriented orthogonal to each of the first inlet and the second inlet. The device may further include a collar coupled to the valve and coupled to the fluid reservoir by a Luer fitting. The collar may be configured to be adjusted to rotatably orient the second inlet to be aligned with an aperture during assembly of the housing.

In certain implementations, the female snap undercut includes a distal sloped surface and a proximal sloped surface directly engaging a complementary proximal sloped surface of the male snap undercut. In certain implementations, the axial position of the central locking feature is closer to the distal end of the female annular projection than a transition between the distal sloped surface and the proximal sloped surface. In certain implementations, the female snap undercut further includes a transition surface between the distal sloped surface and the proximal sloped surface.

In certain implementations, at least one of the female annular projection and the male annular projection defines at least one slot configured to permit deflection of at least one of the female annular projection and the male annular projection for the female snap to be mated with the male snap. Each of the female projection and the male projection may be annular in shape, and the female snap undercut and/or the male snap undercut may be ring-shaped The central locking feature is configured to limit or prevent inward deflection of the male annular projection with the first snap mated with the second snap. The central locking feature may be coaxially disposed within the female annular projection. The central locking feature may be tapered away from the first shell The at least one slot may be two slots extending longitudinally from the distal end of each of the female annular projection and the male annular projection. The two slots may be diametrically opposed to one another. Portions of the female snap undercut and the male snap undercut may be disposed on each side of the two slots of the respective one of the female annular projection and the male annular projection.

### BRIEF DESCRIPTION OF THE DRAWINGS

FIG. 1 is a perspective view of an electrosurgical system.

FIG. 2 is a perspective view of an infusion module.

FIG. 3 is an exploded view of the infusion module.

FIG. 4 is an exploded view of a housing of the infusion module including a female snap and a male snap.

FIG. 5A is a cross-sectional view of the female snap and the male snap of FIG. 4 in a mated configuration.

FIG. 5B is a cross-sectional view of the female snap and the male snap of FIG. 4 in an unmated configuration.

FIG. 6A is a cross-sectional view of alternative snaps in a mated configuration.

FIG. 6B is a cross-sectional view of alternative snaps in an unmated configuration.

FIG. 7A-7D are partial views of the infusion module including a window.

FIGS. 8A-8B are various perspective views of a packaging arrangement for the infusion module.

### DETAILED DESCRIPTION

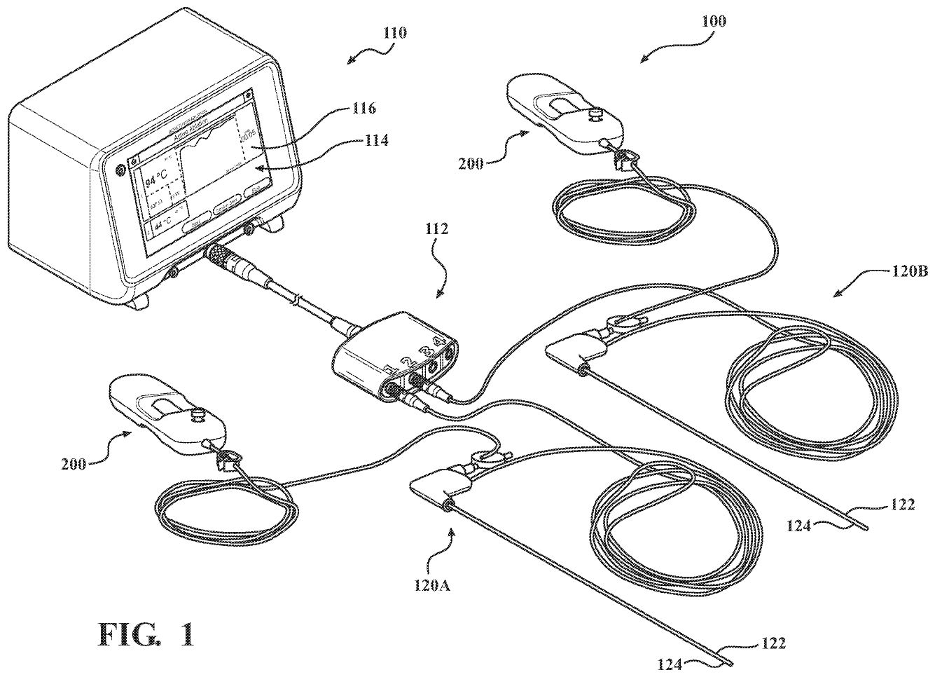

During a radio-frequency (RF) ablation procedure, it is often helpful to deliver a conductive fluid to a treatment site to increase the efficacy of the ablation while also reducing the chance of charring tissue located at the treatment site. As such, an electrosurgical system including an infusion module is provided. Referring to FIG. 1, an electrosurgical system 100 includes an electrosurgical console 110, electrosurgical instrument(s) 120 and, optionally, a cable accessory 112. The cable accessory 112 is configured to be removably coupled with the electrosurgical console 110, and the electrosurgical instrument(s) 120 are configured to be removably coupled with the electrosurgical console 110 and/or the cable accessory 112. The electrosurgical instrument(s) 120 may be a bipolar electrode, for example, a self-grounding bipolar electrode in which proximal and distal electrodes 122, 124 provide a path of electrical current through the tissue to be ablated. Put simply. the electrosurgical instrument 120 may be an ablation probe. The ablation probe may include a thermocouple to provide a temperature measurement of the tissue near the electrode(s) 122, 124. Additional thermocouples may be utilized to monitor soft tissue of points of interest, e.g., spinal canal, to inform the user if the ablation heat migrates to an undesired location. Additionally, the electrosurgical system 100 includes an infusion module 200. The infusion module(s) 200 may direct a fluid (e.g., a liquid) through the electrosurgical instrument(s) 120 to be discharged near proximal and distal electrodes 122, 124. One suitable implementation of the electrosurgical instrument(s) 120 is disclosed in the aformentioned International Publication No. WO2020/198150.

The electrosurgical system 100 is configured to treat the tissue, namely by RF ablation. The electrosurgical console 110 generates electrical energy of a controlled radiofrequency and passes the energy through the electrosurgical instrument 120A, 120B and the tissue, thereby heating the tissue to sufficient temperature to destroy cells of the tissue. The ablation may be carried out through a self-grounding bipolar configuration as detailed in commonly-owned International Publication No. WO 2018/200254, published Nov. 1, 2018, the entire contents of which are hereby incorporated by reference. In certain implementations, the electrosurgical system 100 is utilized to ablate nerves for pain management. In other implementations, the electrosurgical system 100 is utilized to ablate lesions, in particular intraosseous tumors. An exemplary procedure of particular interest is ablation of tumors within the vertebral body. An introducer assembly is deployed through a pedicle of the vertebral body to facilitate access to the vertebral body, and the electrosurgical console 110 applies temperature-controlled, RF energy into the tumor. Nerves associated within or surrounding the tumor may also be ablated to provide pain relief. It should be appreciated that the electrosurgical system 100 of the present disclosure may be utilized to treat intraosseous tumors of long bones, the skull, mandible, ileum, and the like.

The electrosurgical console 110, as shown in FIG. 1, includes a display 114 configured to display a graphical user interface (GUI) 116 for enabling a user, among other actions, to select operating parameters provided by software on the electrosurgical console 110. The display 114, in one example, is a touch screen, enabling selection of digital indicia represented on the display 114 using the location of a touching that may be capacitively sensed on the touch screen. The electrosurgical console 110 generally includes a controller, one or more processors, and memory. Computer-executable instructions may be stored on the memory. for example, within a database of the memory. The instructions are accessible by the processor and executable by the processor to implement various functions of the electrosurgical console 110. An exemplary electrosurgical console 110 may be disclosed in the aformentioned International Publication No. WO 2018/200254.

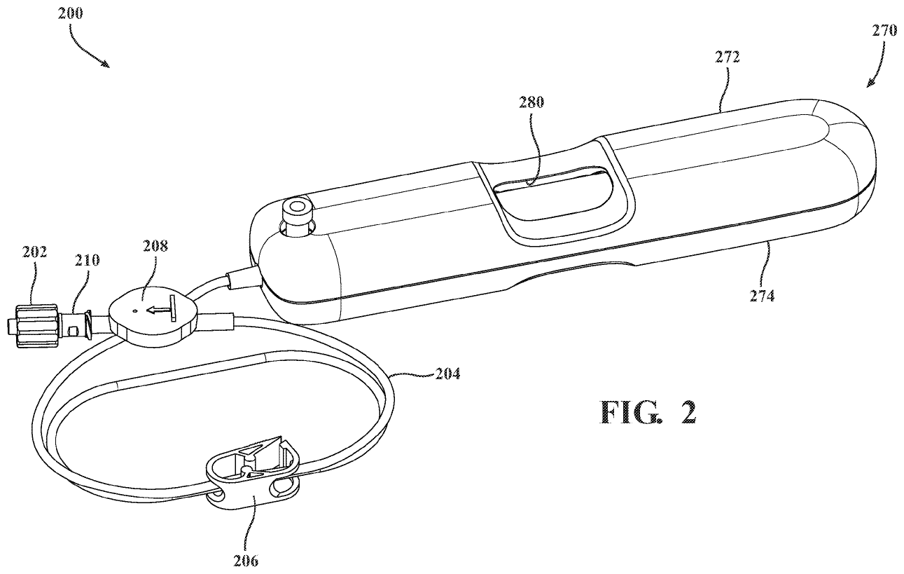

Referring to FIG. 2, the infusion module 200 is shown in more detail. The infusion module 200 may be releasably coupled to the electrosurgical instrument 120A with a fluid coupling 202, such as a Luer lock fitting. When coupled to the electrosurgical instrument 120A. the infusion module 200 is configured to be placed in fluid communication with the electrosurgical instrument 120A. Although the infusion module 200 is shown with a single fluid coupling 202. it is also contemplated to couple the fluid coupling 202 to a split line in order to supply fluid to multiple electrosurgical instruments 120A. Regardless of the number of instruments 120A utilized, the infusion module 200 includes the fluid coupling 202 along with a flexible infusion line 204, a stop clamp 206, a filter and vent assembly 208, and a flow restrictor 210. The infusion module 200 also includes a housing 270 configured to contain the other elements of the infusion module 200. The housing generally consists of a first shell 272 and a second shell 274 with at least one of the first and second shells 272, 274 defining a window 280 through which the user may determine the amount of fluid remaining in the infusion module 200.

The infusion module 200 supplies fluid to the electrosurgical instrument 120A through the flexible infusion line 204. To make sure that only fluid is supplied to the instrument 120A. the filter and vent assembly 208 includes a gas-permeable, liquid-impermeable vent to allow gas to escape before it reaches the fluid coupling 202. In order to control how much fluid is supplied. the stop clamp 206 and the flow restrictor 210 are disposed on the infusion line 204. The stop clamp 206 is capable of stopping the fluid flow entirely (e.g., in a clamped configuration), while the flow restrictor 210 is capable of controlling the amount of fluid supplied when the stop clamp 206 is configured to allow fluid flow (e.g., in an unclamped configuration). The flow restrictor 210 can be configured to constrict fluid flow to a rate between about 0.5 to about 15 milliliters per hour (mL/hour). It should be appreciated that flow rate of fluid output of the infusion module 200 can be modified by using specific examples of the flow restrictor 210 to produce a desired flow rate. Various flow restrictors or flow limiters known to those of skill in the art can be used and for purposes of the present disclosure can be described as any component that is shaped to restrict the flow of fluid to a set flow rate. Some non-limiting examples of such flow restrictors are capillary (e.g., tubing with a predetermined restriction in cross-section to control the flow rate therethrough), while other such flow restrictors use single stage or multi-stage orifice plates to handle high and low flow rates. In many examples, the flow restrictor 210 restricts fluid output to a rate of from about 0.5 to about 15 mL/hour, or about 1 to about 12, mL/hour.

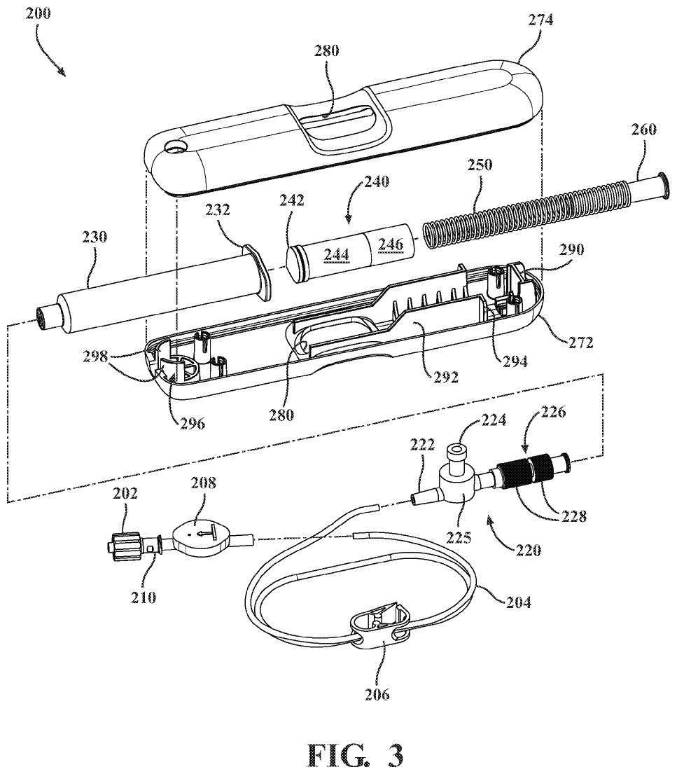

Referring to FIG. 3, the infusion module 200 is shown in an exploded view. In order to couple the infusion module 200 to the infusion line 204, the infusion module 200 includes a port assembly 220 coupled to the infusion line 204 and a fluid reservoir 230. The port assembly 220 includes a port coupling 222, a fill port 224, a port body 225, and a collar assembly 226. In general, the port coupling 222 defines an outlet through which fluid may flow out of the port assembly 220 from the fluid reservoir 230, while the fill port 224 and the collar assembly 226 each include an inlet through which fluid may flow into the port assembly 220. More specifically, fluid may be introduced through the inlet defined by the fill port 224 in order to fill the fluid reservoir 230. During use, the same fluid from the fluid reservoir 230 may flow through the inlet defined by the collar assembly 226 such that the fluid exits the port assembly 220 through the outlet defined by the port coupling 222 and into the infusion line 204. The port assembly 220 is partially housed within the housing 270 of the infusion module 200 and located at a distal end of the fluid reservoir 230. As is best illustrated in FIG. 2, the port coupling 222 extends past the housing 270 of the infusion module 200 and is connected or releasably connected to the flexible infusion line 204. As is also illustrated in FIG. 2, the fill port 224 extends through the second shell 274 of the housing 270 of the infusion module 200. The port assembly 220 advantageously allows for the loading of the infusion module 200 through the inlet defined by the fill port 224 while the infusion module 200 is coupled to the electrosurgical instrument 120A. Further, the infusion module 200 can be reloaded during use if fluid is depleted. Nonetheless, the port assembly 220 also allows the loading of the infusion module 200 when it is not coupled to the instrument 120A.

The infusion module 200 is configured to be releasably connected to a fill source, e.g., a syringe filled with fluid such as saline. Some non-limiting examples of suitable fill source are 5, 6, 7, 8, 9, or 10 mL syringes having a Luer lock tip. The fill source can be filled with a fluid previously drawn from the fluid supply. e.g., an I.V. fluid bag, a sterilized fluid vial, etc. The infusion module 200 of this example can have a total volumetric fluid capacity from about 1 to about 20 mL, or more particularly from about 2 to about 10 mL, or even more particularly from about 2 to about 8 mL. In some examples, the fluid reservoir 230 has a 6 mL capacity. In this example, the port assembly 220 includes the fill port 224 (e.g., a Luer lock coupling, a slip tip coupling, an eccentric tip coupling, a catheter tip coupling, etc.) which releasably connects to a corresponding connector on the fill source and also the port coupling 222 that connects or in some examples releasably connects to the flexible infusion line 204. In order to provide a secure coupling between the fluid reservoir 230 and the infusion line 204, the collar assembly 226 may include both locating and/or length adjusting means. For example, as can be appreciated from FIG. 3, the collar assembly 226 may include at least one adjustment member 228 configured to couple to the port assembly 220 and the fluid reservoir 230 (e.g., by Luer lock fitting) such that adjustment member(s) 228 completes the fluid connection between the fluid reservoir 230 and the port assembly 220. In the depicted embodiment, the collar assembly 226 includes two adjustment members 228 each of which can be rotated relative to the rest of the port assembly 220 to change the length of the collar assembly 226. In addition, the adjustment members 228 allow the port assembly 220 to be rotated relative to the fluid reservoir 230 to make sure that the fill port 224 can exit the exterior surface of the infusion module 200 when assembled.

The fluid reservoir 230 is configured to hold fluid inside of a void defined by the fluid reservoir 230. It should be understood that fluid is used herein to refer to a liquid, but may assume another suitable phase. As is described in more detail below, the fluid reservoir 230 may consist of a clear plastic (e.g., polypropylene) so that the user may look at the fluid reservoir 230 through one of the windows 280 and determine the amount of fluid held inside the void defined by the fluid reservoir 230. In order to provide the pressure necessary to deliver fluid from the fluid reservoir 230 to the electrosurgical instrument 120A, the infusion module 200 also includes a plunger 240 configured to be urged into the void of the fluid reservoir 230 by a biasing element 250. The plunger 240 may be at least partially hollow to accommodate the biasing element 250. In the illustrated embodiment, the biasing element 250 is a spring configured to store potential energy as it is compressed. A support member 260 may be disposed within the biasing element to prevent the biasing element from buckling. For example, where the biasing element 250 is the spring, the support member 260 can be disposed within the spring to prevent the spring from buckling when compressed. In such an embodiment, the biasing element 250 is at least partially disposed within the plunger 240 and around the support member 260. The first shell 272 of the housing 270 may include a shelf 290 configured to abut the biasing element 250 and the support member 260. The shelf 290 provides support for the biasing element 250 when the biasing element 250 is urging the plunger 240 toward and/or into the void of the fluid reservoir 230. Alternatively, the support member 260 may abut the shelf 290 and the biasing element 250 may abut the support member 260. In either case, the shelf 290 provides a fixed surface upon which the biasing element 250 may exert force. It is further contemplated that the second shell 274 of the housing may include the shelf 290 instead of or in addition to the first shell 272.

The plunger 240 generally includes a sealing head 242, a first colored portion 244, and a second colored portion 246. When the fluid reservoir 230 consists of clear plastic, the plunger 240 is visible through the clear plastic when the plunger 240 is disposed within the void defined by the fluid reservoir 230. By extension, the sealing head 242, the first colored portion 244, and the second colored portion 246 may also be visible through the clear plastic of the fluid reservoir 230 depending on how far the plunger 240 has been urged into the void of the fluid reservoir 230. For example, and as expanded on below, the user may look at the fluid reservoir 230 through the window 280 of the infusion module 200 to determine the fluid content of the fluid reservoir 230 based on the visibility of at least one of the sealing head 242, the first colored portion 244, and the second colored portion 246 through the window 280. The plunger 240 may be formed of a colored material such that the plunger 240 is itself colored to include at least one of the colored portions 244, 246. Alternatively, a piece of material, such as colored tape, may be secured to the outside of the plunger 240 so as to define at least one of the colored portions 244. 246. For example, the plunger 240 may be formed of a first colored material (e.g., colored plastic) to define the first colored portion 244, while the second colored portion 246 is created by securing a piece of colored material to the outside of the plunger 240.

In order to keep the fluid reservoir 230 in place, the reservoir 230 may include a collar 232 shaped to abut part of the housing 270. For example, the first shell 272 may include a reservoir guide 292 shaped to receive the fluid reservoir 230. The reservoir guide 292 may include a support surface 294 shaped to abut the collar 232 of the fluid reservoir 230 such that the support surface 294 resists displacement of the fluid reservoir 230 from the forces exerted by the biasing element 250 and the plunger 240. Instead, the collar 232 will be urged against the support surface 294 and the fluid reservoir 230 will be kept in place. Additionally, the first and/or second shells 272, 274 may include an internal barrier to resists forces experienced by the port assembly. For example, the internal barrier may include a port support 296 arranged to support the port assembly 220. The port support 296 is generally shaped to receive the port assembly 220 and may include wings 298 extending from one of the first and second shells 272, 274 toward the other one of the first and second shells 272, 274. In the illustrated embodiment, the wings 298 extend from both the first and second shells 272, 274 and are shaped to abut the port body 225. As the biasing element 250 is applying force to the plunger 240, the plunger 240 applies force to the fluid reservoir 230, and the fluid reservoir 230 applies force to the port assembly 220. The port support 296 and the wings 298 support is arranged to resist this force on the port assembly 220 and to keep the port assembly 220 in the same position relative to the housing 270.

As described above, the first shell 272 of the housing includes the shelf 290 to provide a fixed surface against which the biasing clement 250 stores the potential energy necessary to urge the plunger 240 into the void of the fluid reservoir 230. To ensure that the infusion module 200 provides a consistent volume of fluid over time through the infusion line 204. the biasing element 250 is configured to store a large amount of potential energy. As such, the shelf 290, and thus the housing 270 itself, experiences a significant amount of force from the biasing element 250 when the biasing element 250 is compressed between the shelf 290 and the plunger 240. In order to ensure that the first shell 272 is not separated from the second shell 274 because of said force, the first shell 272 may be secured to the second shell 274 by means of snaps capable of resisting said force.

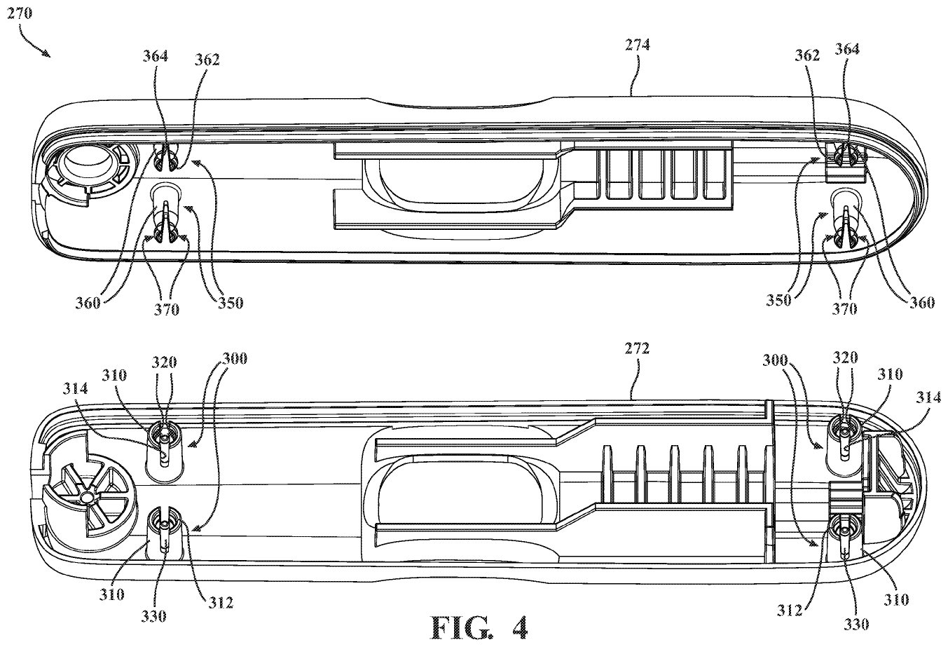

Referring to FIG. 4, the housing 270 of the infusion module 200 is shown with the first shell 272 separated from the second shell 274. The first shell 272 includes a female snap 300 and the second shell 274 includes a male snap 350. The female snap 300 is configured to receive the male snap 350 to fasten the first shell 272 to the second shell 274. As will be appreciated from the figures, each shell 272, 274 may include more than one pair of snaps 300, 350 (e.g., four pairs of snaps) to provide a stronger fastening relationship between the first and second shells 272, 274. If the first shell 272 includes four female snaps 300 and/or the second shell 274 includes four male snaps 350, each male snap 350 is generally identical to the other male snaps 350 and each female snap 300 is generally identical to the other female snaps 300. It is also contemplated for one of the female snaps 300 to be different from the rest of the female snaps 300 and one of the male snaps 350 to be different from the rest of the male snaps 350. For example, one pair of snaps 300, 350 could be configured to provide a stronger fastening relationship compared to the remainder of the snaps 300, 350.

The female snap 300 includes a female annular projection 310, a female snap undercut 320, and a central locking feature 330. The female annular projection 310 extends from the first shell 272 to a distal end 312. In the illustrated embodiment, the female annular projection 310 defines a slot 314 configured to permit deflection of the female annular projection 310 and to better allow the female snap 300 to receive the male snap 350. More specifically, the female annular projection 310 may define a pair of diametrically opposed slots 314, each slot 314 extending longitudinally from the distal end 312 of the female annular projection 310 such that the projection 310 is split into two “sides”. The female annular projection 310 also includes a female snap undercut 320 extending inwardly from the female annular projection 310 so that the female snap 300 can better retain the male snap 350 as described below. The female snap undercut 320 may be located anywhere between the first shell 272 and the distal end 312 of the female annular projection 310.

Similar to the female snap 300, the male snap 350 includes a male annular projection 360 and a male snap undercut 370. The male annular projection 360 extends from the second shell 274 to a distal end 362. In the illustrated embodiment, the male annular projection 360 defines a slot 364 configured to permit deflection of the male annular projection 360 and to better allow the male snap 350 to be received by the female snap 300. More specifically, the male annular projection 360 may define a pair of diametrically opposed slots 364, each slot 364 extending longitudinally from the distal end 362 of the male annular projection 360 such that the projection 360 is split into two “sides”. The male annular projection 360 also includes a male snap undercut 370 extending outwardly from the male annular projection 360 and located at the distal end 362 of the male annular projection 360. The male snap undercut 370 is configured to engage the female snap undercut 320 when the snaps 300, 350 are mated with one another.

The female snap 300 further includes the central locking feature 330 disposed coaxially within the female annular projection 310. As described in more detail below, the central locking feature 330 is configured to be received by the male annular projection 360 when the snaps 300, 350 are mated with one another and to resist inward deformity of the male annular projection 360. The central locking feature 330 extends from the first shell 272 to an axial position closer to the distal end 312 of the female annular projection 310 than the female snap undercut 320. In other words, the central locking feature 330 generally ends at an axial position between the distal end 312 of the female annular projection 310 and the snap undercut 320. It is also contemplated for the central locking feature 330 to be at least as long as the female annular projection 310 and end at an axial position substantially equal to or greater than that of the distal end 312 of the female annular projection 310. Regardless of the relative length of the central locking feature 330, the male annular projection 360 is configured to receive the central locking feature 330 and thus the projection 360 must be long enough to accommodate the central locking feature 330 without inhibiting the mateability of the snaps 300, 350. In the illustrated embodiment, the central locking feature 330 includes a sloped distal end 332 to help locate the male snap 350 as it is urged toward the female snap 300.

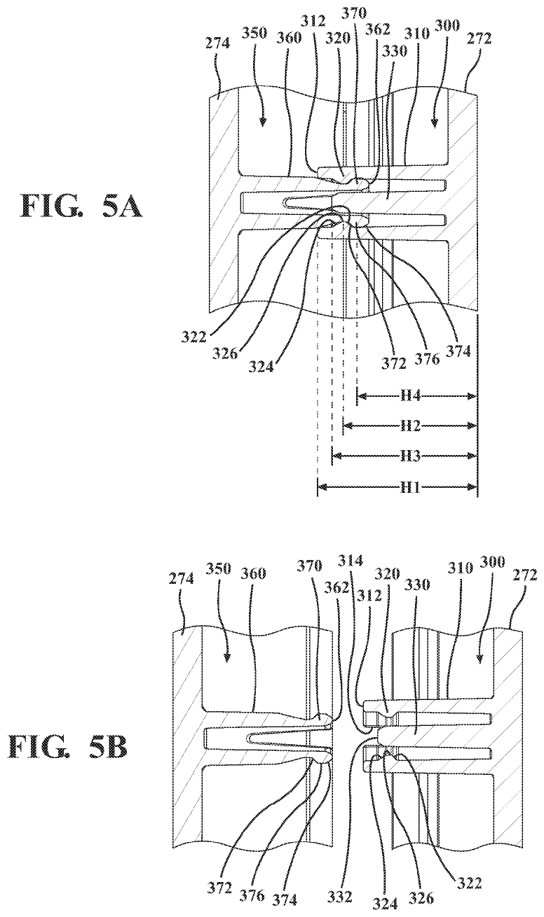

Referring to FIGS. 5A and 5B, the snaps 300, 350 are shown in a mated configuration in FIG. 5A and an unmated configuration in FIG. 5B. As will be appreciated from the figures, the female annular projection 310 includes the female snap undercut 320 configured to engage the male snap undercut 370 when the snaps 300, 350 are in the mated configuration of FIG. 5A. To that end, the female snap undercut 320 may include a proximal sloped surface 322, a distal sloped surface 324, and a transition surface 326 extending between the proximal and distal sloped surfaces 322, 324 of the female snap undercut 320. Similarly, the male snap undercut 370 may include a proximal sloped surface 372, a distal sloped surface 374, and a transition surface 376 extending between the proximal and distal sloped surfaces 372, 374 of the male snap undercut 370. This way, the snap undercuts 320, 370 are substantially trapezoidal in shape. Although the illustrated embodiment shows the transition surfaces 326, 376 extending between the respective sloped surfaces 322, 324 and 372, 374, the transition surfaces 326, 376 may not exist at all. Instead, the proximal sloped surfaces 322, 372 may directly meet the respective distal sloped surface 324, 374 such that the snap undercuts 320, 370 are substantially triangular in shape. Regardless of the configuration. the snap undercuts 320, 370 generally extend radially from the annular projections 310, 360 such that the snap undercuts 320, 370 are ring-shaped.

As the female annular projection 310 is urged toward and into the mated configuration with the male annular projection 360, the distal sloped surface 374 of the male snap undercut 370 abuts at least one of the distal sloped surface 324 of the female snap undercut 320, and/or a sloped distal end 332 of the central locking feature 330. These two sloped surfaces 324, 374 form a V-shaped receiving channel toward the distal end 312 of the female annular projection 310. This V-shaped receiving channel helps locate the male annular projection 360 into the female annular projection 310 as the male snap 350 is urged toward the female snap 300.

Once the snaps 300, 350 are in the mated configuration of FIG. 5A, the proximal sloped surface 322 of the female snap undercut 320 abuts the proximal sloped surface 372 of the male snap undercut 370. If the snaps 300, 350 are then urged away from one another in an attempt to return the snaps 300, 350 to the unmated configuration, the contact between the female snap undercut 320 and the male snap undercut 370 causes the female annular projection 310 to substantially prevent the male annular projection 360 from being removed from the female annular projection 310. Thus, for the male annular projection 360 to be removed from the female annular projection 310, at least one the annular projections 310, 360 must deflect outwardly or inwardly, respectively. For example, if the male annular projection 360 were to deflect inwardly, the male snap undercut 370 may no longer abut the female snap undercut 320 and the male annular projection 360 may be removed from the female annular projection 310. Alternatively, if the female annular projection 310 were to deflect outwardly, a similar result would occur.

With the above in mind, it is apparent that the fastening strength of the snaps 300, 350 is dependent on the amount of force required to cause at least one of the female and/or male annular projections 310, 360 to deflect outwardly and/or inwardly, respectively. Therefore, the fastening strength of the snaps 300, 350 may be influenced by the material from which the snaps 300, 350 are created, the shape of the annular projections 310, 360 (e.g., the length of the slots 314, 364), the shape of the undercuts 320, 370, and/or a locking element limiting the deflection of at least one of the annular projections 310, 360. In the illustrated embodiment of FIGS. 3-5B, the locking element is the central locking feature 330 which is configured to limit the inward deflection of the male annular projection 360. Therefore, because the male annular projection 360 is restricted from deflecting inward, the female annular projection 310 must deflect outward to allow the snaps 300, 350 to move from the mated configuration to the unmated configuration.

In order for the central locking feature 330 to limit the inward deflection of the male annular projection 360, the male snap undercut 370 must be urged against the central locking feature 330 and the female snap undercut 320 at the same time. More specifically, as the male snap 350 is urged away from the female snap 300, the central locking feature 330 must resist inward deflection of the male annular projection 360 before the proximal sloped surface 372 of the male snap undercut 370 moves out of contact with the proximal sloped surface 322 of the female snap undercut 320. Therefore, the central locking feature 330 generally extends from the first shell 272 to an axial height at least as high as (or higher than) the female snap undercut 320. In this way the male snap undercut 370 contacts the central locking feature 330 before the proximal sloped surface 372 of the male snap undercut 370 moves out of contact with the proximal sloped surface 322 of the female snap undercut 320. Further, the central locking feature 330 must be of a sufficient radius such that the male annular projection 360 cannot deflect inward to the point that the proximal sloped surface 372 of the male snap undercut 370 moves out of contact with the proximal sloped surface 322 of the female snap undercut 320 before the male annular projection 360 contacts the central locking feature 330.

Another way to illustrate the relationship between the snaps 300, 350 is with a series of heights relative to the first shell 272 as shown in FIG. 5A. For example, a first height H1 may represent the length of the female annular projection 310 from the first shell, a second height H2 may represent the distance between the first shell 272 and the female snap undercut 320. a third height H3 may represent the distance between the first shell 272 and the distal end 332 of the central locking feature 330, and a fourth height H4 may represent the distance between the first shell 272 and the male snap undercut 370 when the male snap 350 is mated with the female snap 300. In the figures, the second height H2 is aligned with the transition surface 326 of the female snap undercut 320 and the fourth height H4 is aligned with the transition surface 376 of the male snap undercut 370. Alternatively, if the proximal and distal sloped surfaces 322, 324 of the female snap undercut 320 come together without the transition surface 326 in between, the second height H2 may be aligned with the transition between the proximal sloped surface 322 and the distal sloped surface 324. Similarly, if the proximal and distal sloped surfaces 372, 374 of the male snap undercut 370 come together without the transition surface 376 in between, the fourth height H4 may be aligned with the transition between the proximal sloped surface 372 and the distal sloped surface 374. Generally, the first height H1 is the largest, followed by the third height H3, followed by the second height H2, and with the fourth height H4 being the smallest of the heights H1, H2, H3, H4. However, the first height H1 may be substantially equal to the third height H3.

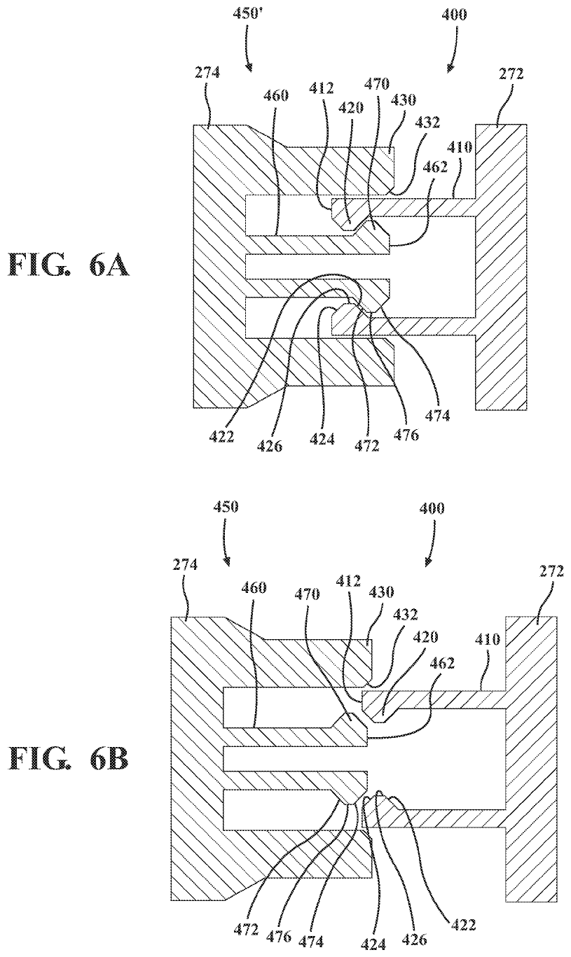

Referring to FIGS. 6A and 6B, an alternative snap embodiment is shown which includes a female snap 400 and a male snap 450. These alternative snaps 400, 450 are substantially similar to snaps 300, 350, however, the central locking feature 330 has been replaced by an outer locking feature 430. Thus, most of the elements of the earlier discussed snaps 300, 350 are present in the alternative snaps 400, 450 and similar elements are referenced by the same later two digits with the first digit changed from 3 to 4 (e.g., 300 to 400 and 350 to 450). The alternative snaps 400, 450 are shown in the mated configuration in FIG. 6A and the unmated configuration in FIG. 6B.

Similar to female snap 300, the female snap 400 includes a female annular projection 410 and a female snap undercut 420. The female annular projection 410 extends from the first shell 272 to a distal end 412. Although not shown in the figures. the female annular projection 410 defines a slot 414 configured to permit deflection of the female annular projection 410 and to better allow the female snap 400 to receive the male snap 450. More specifically, the female annular projection 410 may define a pair of diametrically opposed slots 414, each slot 414 extending longitudinally from the distal end 412 of the female annular projection 410. The female annular projection 410 also includes a female snap undercut 420 extending inwardly from the female annular projection 410 so that the female snap 400 can better retain the male snap 450. The female snap undercut 420 may be located anywhere between the first shell 272 and the distal end 412 of the female annular projection 410.

Similar to the male snap 350, the male snap 450 includes a male annular projection 460 and a male snap undercut 470. The male annular projection 460 extends from the second shell 274 to a distal end 462. Although now shown in the figures, the male annular projection 460 defines a slot 464 configured to permit deflection of the male annular projection 460 and to better allow the male snap 450 to be received by the female snap 400. More specifically, the male annular projection 460 may define a pair of diametrically opposed slots 464. each slot 464 extending longitudinally from the distal end 462 of the male annular projection 460. The male annular projection 460 also includes a male snap undercut 470 extending outwardly from the male annular projection 460 and located at the distal end 462 of the male annular projection 460. The male snap undercut 470 is configured to engage the female snap undercut 420 when the snaps 400, 450 are mated with one another.

As an alternative to the central locking feature 330 of the snaps 300, 350 depicted in FIGS. 5A and 5B, the male snap 450 of FIGS. 6A and 6B further includes the outer locking feature 430. As described in more detail below, the outer locking feature 430 is configured to cooperate with the male annular projection 460 to receive the female annular projection 410 when the snaps 400, 450 are mated with one another and to resist outward deformity of the female annular projection 410. The outer locking feature 430 extends from the second shell 274 to an axial position at least substantially equal to the distal end 462 of the male annular projection 460. In the illustrated embodiment, the outer locking feature 430 includes a sloped distal end 432 to help locate the female snap 400 as it is urged toward the male snap 450.

Similar to the earlier snaps 300, 350, the female annular projection 410 includes the female snap undercut 420 configured to engage the male snap undercut 470 when the snaps 400, 450 are in the mated configuration of FIG. 6A. To that end, the female snap undercut 420 may include a proximal sloped surface 422, a distal sloped surface 424, and a transition surface 426 extending between the proximal and distal sloped surfaces 422, 424 of the female snap undercut 420. Similarly, the male snap undercut 470 may include a proximal sloped surface 472, a distal sloped surface 474, and a transition surface 476 extending between the proximal and distal sloped surfaces 472, 474 of the male snap undercut 470. As the female annular projection 410 is urged toward and into the mated configuration with the male annular projection 460, the distal sloped surface 424 of the female snap undercut 420 abuts at least one of the distal sloped surface 474 of the male snap undercut 470 and/or a sloped distal end 432 of the outer locking feature 430. These two sloped surfaces 424, 474 form a V-shaped receiving channel toward the distal end 412 of the male annular projection 460. This V-shaped receiving channel helps locate the male annular projection 460 into the female annular projection 410 as the male snap 400 is urged toward the female snap 450.

Once the snaps 400, 450 are in the mated configuration of FIG. 6A, the proximal sloped surface 422 of the female snap undercut 420 abuts the proximal sloped surface 472 of the male snap undercut 470. If the snaps 400, 450 are then urged away from one another in an attempt to return the snaps 400, 450 to the unmated configuration, the contact between the female snap undercut 420 and the male snap undercut 470 causes the female annular projection 410 to substantially prevent the male annular projection 460 from being removed from the female annular projection 310. Thus, the only way for the male annular projection 460 to be removed from the female annular projection 410 is for at least one the annular projections 410, 460 to deflect outwardly or inwardly, respectively. For example, if the male annular projection 460 were to deflect inwardly, the male snap undercut 470 may no longer abut the female snap undercut 420 and the male annular projection 460 may be removed from the female annular projection 410. Alternatively, if the female annular projection 410 were to deflect outwardly, a similar result would occur.

In order for the outer locking feature 430 to limit the outward deflection of the female annular projection 410. the female snap undercut 420 must be urged against the outer locking feature 430 and the male snap undercut 470 at the same time. More specifically, as the male snap 450 is urged away from the female snap 400, the outer locking feature 430 must resist outward deflection of the female annular projection 410 before the proximal sloped surface 422 of the female snap undercut 420 moves out of contact with the proximal sloped surface 472 of the male snap undercut 470. Therefore, the outer locking feature 430 generally extends from the second shell 272 to an axial height at least as high as (or higher than) the male snap undercut 420. This way the female snap undercut 420 contacts the outer locking feature 430 before the proximal sloped surface 422 of the female snap undercut 420 moves out of contact with the proximal sloped surface 472 of the male snap undercut 470. Further, the outer locking feature 430 must be close enough to the male annular projection 460 such that the female annular projection 410 cannot deflect outward to the point that the proximal sloped surface 422 of the female snap undercut 420 moves out of contact with the proximal sloped surface 472 of the male snap undercut 470 before the female annular projection 410 contacts the outer locking feature 430.

The housing 270 and thus the snaps 300, 350, 400, 450 may be formed by various molding techniques, such as injection molding. By extension, the housing 270 and the snaps 300, 350, 400, 450 may be formed of a plastic material compatible with the selected forming process. Two such classes of materials that may be used are thermoplastics and thermosetting plastics. It is further contemplated to form the snaps 300, 350, 400, 450 of a material different from the remainder of the housing 270, or even to form the female snap 300, 400 of a material different from the male snap 350, 450. For example, since the locking features 330, 430 prevent the male annular projection 360 from deflecting inward and the female annular projection 410 from deflecting outward, these projections 360, 410 may not need to be formed of a plastic as strong as the complement projections 310, 460.

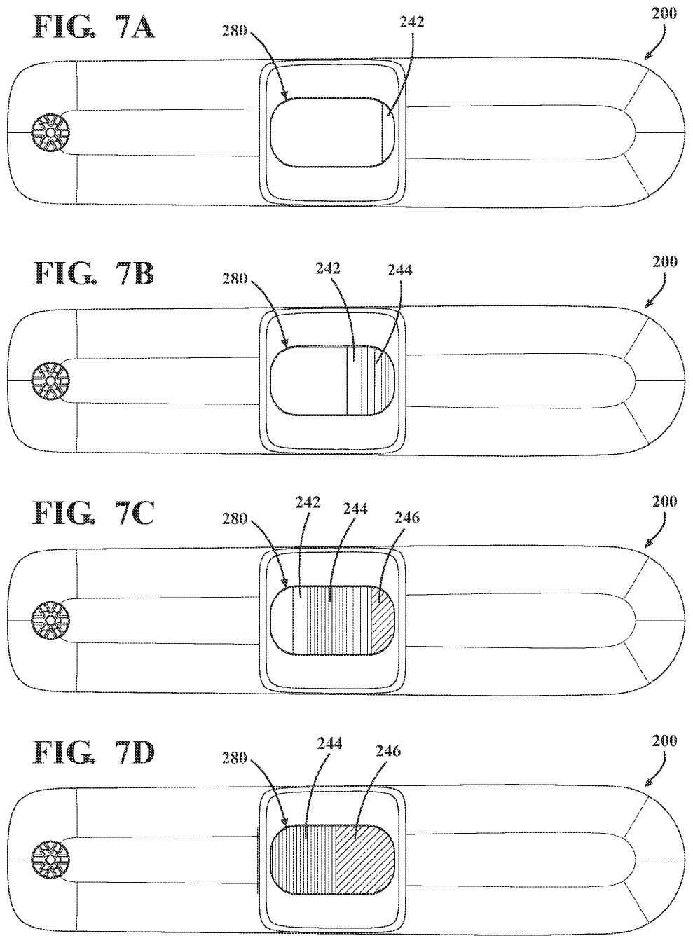

Referring to FIGS. 7A-7D, partial views of the infusion module 200 including the window 280 are shown. These views focus on the elements of the infusion module 200 which are viewable through the window 280 while the fluid reservoir 230 is filled with various amounts of fluid-these viewable elements are the sealing head 242, the first colored portion 244, and the second colored portion 246. In effect, this allows the user to look at the fluid reservoir 230 through the window 280 of the infusion module 200 and to determine the fluid content of the fluid reservoir 230 based on the visibility of at least one of the sealing head 242, the first colored portion 244, and the second colored portion 246 through the window 280. In addition, since the fluid reservoir 230 is substantially clear as described above, the fluid contained therein may also be viewable through the window 280. Although the fluid reservoir 230 is substantially clear, the reservoir 230 may also include incremental hash marks denoting how much fluid remains. For example, the fluid reservoir 230 may include 6 larger hash marks denoting 1 mL increments and 4 smaller hash marks denoting ⅕ mL increments.

Referring to FIG. 7A. the infusion module 200 is shown with the fluid reservoir 230 filled at substantially maximum capacity (e.g., filled with 6 mL of fluid out of 6 mL total capacity). In this state, the sealing head 242 is viewable through the window 280 while the first and second colored portions 244, 246 are not viewable through the window 280. In one example, the infusion module 200 may reach this state after the user fills the fluid reservoir 230 to maximum capacity but before the infusion module 200 is used to provide fluid to the treatment site.

FIG. 7B shows the infusion module 200 with the fluid reservoir 230 filled to equal to or more than a third of maximum capacity but less than maximum capacity (e.g., filled with 2-5.9 mL of fluid out of 6 mL total capacity). In this state, the sealing head 242 and the first colored portion 244 are both viewable through the window 280 while the second colored portion 246 is not viewable through the window 280. In one example, the infusion module 200 may reach this state after the infusion module 200 provides between 0.1 and 3 mL of fluid to the treatment site.

Referring to FIG. 7C, the infusion module 200 is shown with the fluid reservoir 230 filled to less than a third of maximum capacity (e.g., filled with equal to or less than 2 mL of fluid out of 6 mL total capacity). In this state, the sealing head 242 and the first and second colored portions 244, 246 are all viewable through the window 280. Additionally, only a small amount of the second colored portion 246 is viewable through the window 280. In one example, the infusion module 200 may reach this state after the infusion module 200 provides between at least 4 mL but less than 6 mL of fluid to the treatment site.

Lastly, FIG. 7D shows the infusion module 200 with the fluid reservoir 230 substantially empty (e.g., filled with around 0 mL of fluid out of 6 mL total capacity). In this state, the first and second colored portions 244, 246 are both viewable through the window 280. A larger amount of the second colored portion 246 is viewable through the window 280 when the fluid reservoir 230 is empty compared to when the fluid reservoir 230 is filled to less than a third of maximum capacity. Although the sealing head 242 is no longer viewable through the window in FIG. 7D, it is contemplated that the sealing head 242 may be viewable (or not) when the fluid reservoir 230 is substantially empty. In one example, the infusion module 200 may reach this state after the infusion module 200 provides 6 mL of fluid to the treatment site. Additionally, as described below, the infusion module 200 may be shipped with an empty fluid reservoir 230 and the first and second colored portions 244, 246 may be viewable through the window 280 prior to the use of the infusion module 200.

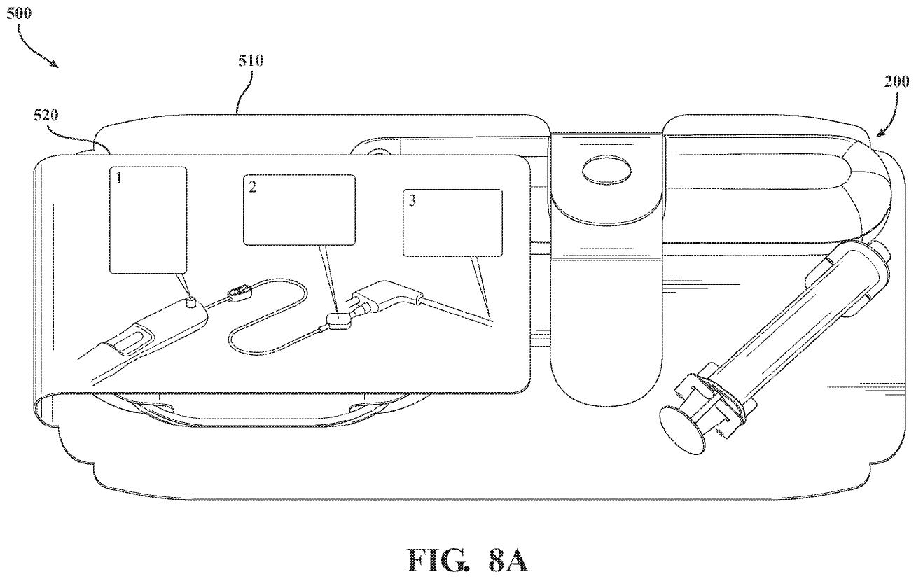

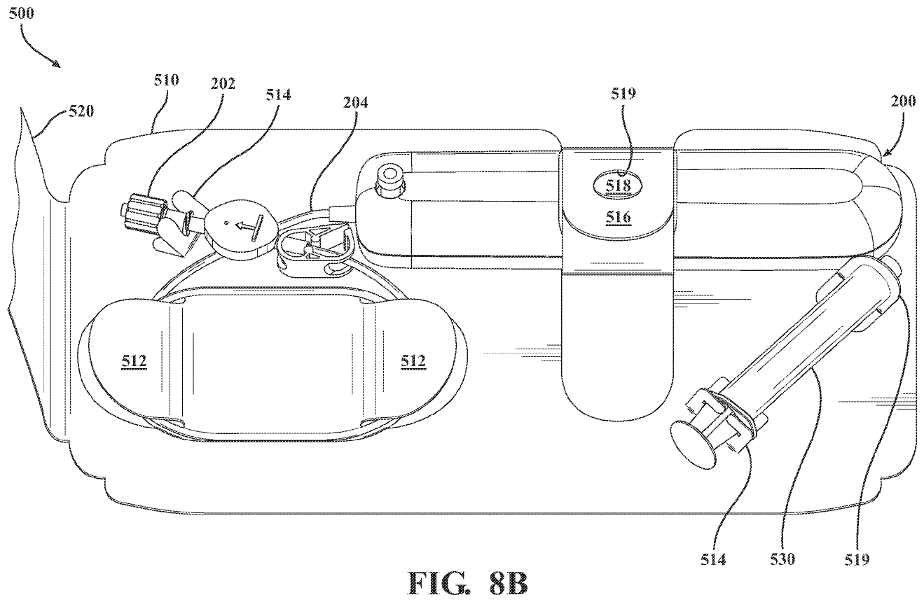

Referring to FIGS. 8A and 8B, a packaging assembly 500 for the infusion module 200 is shown. The packaging assembly 500 is configured to allow the manufacturer to ship the infusion module 200 and other associated elements in a secure and organized manner. To that end, the packaging assembly 500 generally includes an attachment surface 510 configured to removably couple the infusion module 200 and the other associated elements to the packaging assembly 500, and an informational surface 520 optionally coupled to the attachment surface 510 and configured to present information to the user. The packaging assembly 500 may be made of a pliable paper and/or plastic material(s) and may consist of a single piece or multiple pieces. In the illustrated embodiment, the packaging assembly 500 is a single piece of material with the attachment surface 510 being substantially planar and connected to the informational surface 520 by a bend in the material.

Referring specifically to FIG. 8A, the information surface 520 is shown as a folded-over piece of the packaging assembly 500 and formed of the same piece of material as the attachment surface 510. As such, the manufacturer does not need to separately include a supplemental informational flyer or packet as is usually done when shipping a product with informational material. Moreover, the information surface 520 is available in the sterile field for the user, in contrast to typical instructions for use that not sterile and cannot easily be accessed by the user. In FIG. 8A, the informational surface 520 is shown folded over a portion of the attachment surface 510 such that some of the attachment surface 510 is hidden behind the informational surface 520. This way, the user is directed to information contained on the informational surface 520 prior to removing the elements attached to the attachment surface 510 (e.g., the infusion module 200). The informational surface 520 generally contains instructions meant to guide the user in using the infusion module 200 along with its associated elements. In the figures, for example, the instructions include: (1) filling the infusion module 200 with a fluid via the fill port 224, (2) connecting the infusion module 200 to the electrosurgical instrument 120A via the fluid coupling 202, and (3) priming the infusion module 200 by waiting for a certain length of time for fluid to start flowing out of the electrosurgical instrument 120A. It is further contemplated to include additional/different instructions, and/or additional/different information on the informational surface 520. The informational surface 520 may further include instructions/information on the other side of the material such that the user is presented more information upon unfolding the informational surface 520 relative to the attachment surface 510. As one example, one side of the informational surface 520 may contain instruction on how to read the information present through the window 280 of the infusion module 200 as described above in reference to FIGS. 7A-7D. Other examples are contemplated.

Referring specifically to FIG. 8B, the attachment surface 510 is shown in more detail and the informational surface 520 is unfolded such that the attachment surface 510 is no longer hidden behind the informational surface 520. As shown, the attachment surface 510 includes features configured to removable couple at least the infusion module 200, the infusion line 204, the fluid coupling 202, and a fill source 530 to the attachment surface 510. These features may include cutouts in the attachment surface 510 to form locating features 512, securing features 514, and wrapping features 516 including tabs 518. The features 512, 514, 516 may be formed by cutting the material of the attachment surface 510 with a sharp object and then folded the material over to form the features 512, 514, 516. In the exemplary embodiment shown in the figures, the infusion module 200 is removable coupled to the attachment surface 510 via the wrapping feature 516. The wrapping feature 516 is formed from material cut out from the attachment surface 510 (although still connected to the attachment surface 510 and at least a portion of the cutout) and is configured to be wrapped around the infusion module 200. The wrapping feature 516 includes the tab 518 and a receiving cutout 519. As the wrapping feature 516 is wrapped around the infusion module 200, the tab 518 is directed into the receiving cutout 519. One the tab 518 is received by the receiving cutout 519, the wrapping feature 516 is held securely about the infusion module 200. Once desired, the wrapping feature 516 may be pulled away from the infusion module 200 to cause the tab 518 to be removed from the receiving cutout 519 such that the infusion module 200 may be removed from the attachment surface 510. The infusion line 204 is removably coupled to the attachment surface 510 by locating features 512 formed as cutout potions of the attachment surface 510. The infusion line 204 is wrapped around the locating features 512 and the fluid coupling 202 is then removably secured to the attachment surface by the securing feature 514. The securing features 514, similar to the other features 512, 516, are also cut out pieces of material from the attachment surface 510 while still being connected to the attachment surface 510 by at least some material. The fill source 530 is also removably secured to the attachment surface 510 by the securing features 514. It is further contemplated that the elements 200, 202, 204, 530 may be secured to the attachment surface 510 via other means. Additionally, the fill source 530 may be the external fill source as described above.

In order to reduce strain on the various components of the infusion module 200, the infusion module 200 is, in many examples, assembled and provided with a minimal amount of compression on the biasing element 250 and minimal to no volumetric fluid capacity in the fluid reservoir 230. This way, the infusion module 200 is not packaged with fluid that may escape the fluid reservoir 230 and/or unnecessary strain on the shelf 290 from the biasing element 250. After the user removes the infusion module 200 from the packaging, the user is instructed on how to use the infusion module 200 by the informational surface 520. For example, to load the infusion module 200, the fill source 530 is connected to the fill port 224 via Luer lock or a similar connection mechanism and fluid is injected into the infusion module 200. During injection, the fluid reservoir 230 is filled with fluid and the biasing element 250 compresses further as the volumetric fluid capacity of the fluid reservoir 230 increases to accommodate fluid injected. As the volumetric fluid capacity of the fluid reservoir 230 increases, the plunger 240 is moved out of the fluid reservoir 230 and toward the shelf 290. Further, as the volumetric fluid capacity of the fluid reservoir 230 increases, the biasing element 250 is further compressed. Stated simply, loading the infusion module 200 simultaneously fills the fluid reservoir 230 and stores potential energy in the biasing element 250.

Once the infusion module 200 is loaded and connected to the electrosurgical instrument 120A via the fluid coupling 202, the fluid reservoir 230 is in fluidic communication with the infusion line 204 and the fluid coupling 202, and the biasing element 250 is configured to release the potential energy and actuate the plunger 240 to discharge fluid from the fluid reservoir 230, into the infusion line 204, through the fluid coupling 202, into the electrosurgical instrument 120A, and finally to the treatment site.

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described. Certain inventive aspects are disclosed according to the following exemplary clauses:
Clause 1—A method of monitoring delivery of a liquid during an ablation procedure with a medical device including a housing defining a window, a fluid reservoir within the housing, and a plunger biased within the fluid reservoir to discharge the liquid, the method comprising: viewing a sealing head of the plunger through the window with the fluid reservoir containing a first volume of the liquid; operating the medical device to discharge a portion of the liquid from the fluid reservoir; viewing a first colored portion of the plunger through the window with the fluid reservoir containing a second volume of the liquid that is less than the first volume; further operating the medical device to discharge another portion of the liquid from the fluid reservoir; and viewing a second colored portion of the plunger through the window with the fluid reservoir containing a third volume of the liquid that is less than the first volume and the second volume.
Clause 2—The method of clause 1, further comprising viewing the liquid through the window and through the fluid reservoir with the fluid reservoir containing the first volume of the liquid.
Clause 2—The method of clause 1 or 2, further comprising viewing only the sealing head through the window as being indicative that the fluid reservoir is substantially filled with the liquid.
Clause 3—The method of clause 1 or 2, further comprising viewing each of the sealing head, the first colored portion, and the second colored portion as being indicative of a low volume of the liquid in which less than approximately 35% of a filled volume of the liquid remains within the fluid reservoir.
Clause 4—The method of clause 1 or 2, further comprising viewing only the first colored portion and the second colored portion as being indicative that the fluid reservoir is substantially empty.
Clause 5—The method of any one of clauses 1-4, wherein the fluid reservoir is packaged as empty, the method further comprising filling the medical device by directing the liquid from a fill source through a fill port and into the fluid reservoir.
Clause 6—The method of clause 5, further comprising priming the medical device by operating the device for a predetermined duration prior to commencing the ablation procedure.
Clause 7—The method of clause 6, wherein the steps of filling the medical device and priming the device are printed on packaging to which the device and an external source are coupled.

1. A medical device for delivering a liquid to an electrosurgical instrument, the device comprising:
a housing defining a window;
a fluid reservoir disposed within the housing and configured to receive the liquid, wherein at least a portion of the fluid reservoir is transparent;
a plunger movably disposed within the fluid reservoir and comprising a first colored portion and a second colored portion being of a different color than the first colored portion; and
a spring disposed within the housing and configured to be biased to cause the plunger to discharge the liquid to the electrosurgical instrument,
wherein the first colored portion of the plunger is viewable through the window with the fluid reservoir containing a first volume of the liquid, and wherein movement of the plunger from the bias of the spring is configured to expose the second colored portion of the plunger within the window as the fluid reservoir discharges at a least a portion of the liquid to contain a second volume of the liquid that is less than the first volume.

2. The medical device of claim 1, wherein the second colored portion is not visible through the window with the fluid reservoir containing the first volume of the liquid.

3. The medical device of claim 1, wherein the liquid is viewable through the window with the fluid reservoir containing the first volume of the liquid.

4. The medical device of claim 1, wherein the plunger further comprises a sealing head being of another different color than the first colored portion and the second colored portion, wherein the sealing head is viewable through the window with the fluid reservoir containing a third volume of the liquid that is greater than the first volume, and, optionally, wherein the third volume is indicative that the fluid reservoir is substantially filled with the liquid.

5. The medical device of claim 1, wherein the plunger further comprises a support member coaxially disposed within the spring and engaging an internal shelf of the housing.

6. A medical device for delivering a liquid to an electrosurgical instrument, the device comprising:
a housing defining a window;
a fluid reservoir disposed within the housing and configured to receive the liquid, wherein at least a portion of the fluid reservoir is transparent;
a plunger movably disposed within the fluid reservoir and comprising a first colored portion and a second colored portion being of a different color than the first colored portion; and
a spring disposed within the housing and configured to be biased to cause the plunger to discharge the liquid to the electrosurgical instrument,
wherein the second colored portion of the plunger is viewable through the window to be indicative of low volume of the liquid in which less than approximately 35% of a filled volume of the liquid remains within the fluid reservoir.

7. The medical device of claim 6, wherein the second colored portion is not visible through the window with the fluid reservoir being substantially filled with the liquid.

8. The medical device of claim 6, wherein the liquid is viewable through the window with the fluid reservoir being substantially filled with the liquid.

9. The medical device of claim 1, wherein the first colored portion and the second color portion spanning a length of the window is indicative that the fluid reservoir is substantially empty.

10. The medical device of claim 1, wherein the window is oblong.

11. The medical device of claim 1, wherein a length of the window corresponds to approximately six milliliters of the liquid being viewable within the fluid reservoir.

12. The medical device of claim 1, wherein the fluid reservoir is devoid of volume markings.

13. The medical device of claim 1, wherein the plunger is formed from a material of a same color as the first colored portion, and wherein the second colored portion is coupled to the plunger.

14. The medical device of claim 1, further comprising a port assembly comprising a collar assembly coupled to an outlet the fluid reservoir, and a fill port extending through the housing and configured to receive liquid from an external fluid source for loading or reloading the fluid reservoir.

15. The medical device of claim 14, wherein the port assembly is engaging a second internal barrier of the housing so as to resist movement of the fluid reservoir from forces from the plunger being biased by the spring.

16-41. (canceled)

42. The medical device of claim 6, wherein the first colored portion and the second color portion spanning a length of the window is indicative that the fluid reservoir is substantially empty.

43. The medical device of claim 6, further comprising a port assembly a port assembly comprising a collar assembly coupled to an outlet the fluid reservoir, and a fill port extending through the housing and configured to receive liquid from an external fluid source for loading or reloading the fluid reservoir.

44. The medical device of claim 43, wherein the port assembly engages a second internal barrier of the housing so as to resist movement of the fluid reservoir from forces from the plunger being biased by the spring.

45. A medical device for delivering a liquid to an electrosurgical instrument, the device comprising:
a housing defining a window;
a fluid reservoir disposed within the housing and configured to receive the liquid, wherein at least a portion of the fluid reservoir is transparent;
a plunger movably disposed within the fluid reservoir; and
a spring disposed within the housing and configured to be biased to cause the plunger to discharge the liquid to the electrosurgical instrument,
a port assembly comprising a collar assembly coupled to an outlet the fluid reservoir, a fill port extending through the housing and configured to receive liquid from an external fluid source for loading or reloading the fluid reservoir, and a port coupler configured to be coupled to and discharge the liquid to an infusion line.

46. The medical device of claim 45, wherein the fill port is orthogonal to each of the collar assembly and the port coupler.
